(19) United States
(12) Patent Application Publication
(10) Pub. No.: US 2007/0275382 A1
(43) Pub. Date: Nov. 29, 2007

(54) Biomarkers for the Efficacy of Somatostatin Analogue Treatment

(76) Inventor: Muriel Saulnier, Millburn, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,778

(22) PCT Filed: Nov. 24, 2004
(86) PCT No.: PCT/EP04/13350

§ 371 Date: Mar. 23, 2007

Related U.S. Application Data
(60) Provisional application No. 60/525,079, filed on Nov. 25, 2003.

(51) Int. Cl. A61K 38/31; C12Q 1/68

(52) U.S. Cl. 435/6; 514/10

(57) ABSTRACT

Gene expression assays were performed using tissues of monkeys treated with the somatostatin analogue pasireotide at sub-therapeutic dose for 14 days. The assays were analyzed to identify the modes of actions of pasireotide with relationships to therapeutic applications. The effects on the growth hormone/IGF-1 and glucagon/insulin axes were reflected in transcript level changes in several organs. The expressed genes are useful as surrogate markers of the biological activity of pasireotide, especially the findings for IGF-2 in the pituitary and kidneys.

### FIELD OF THE INVENTION

This invention relates generally to the analytical testing of tissue samples in vitro, and more particularly to aspects of gene expression profiling concerning growth regulation.

### BACKGROUND OF THE INVENTION

Somatostatin (SST-14; SRIF) is a cyclic tetradecapeptide hypothalamic hormone containing a disulfide bridge between position 3 and position 14. See, U.S. Pat. No. 6,225,284, incorporated herein by reference. Somatostatin also occurs as a 28 amino acid peptide (SST-28). Among its mechanisms, somatostatin inhibits the release of growth hormone (GM) and thyroid-stimulating hormone (TSH), thus inhibiting the release of insulin and glucagon, and reducing gastric secretion. Metabolism of somatostatin by aminopeptidases and carboxypeptidases leads to a short duration of action. Somatostatin binds to five distinct high affinity membrane associated receptor (SSTR) subtypes with relatively high affinity for each subtype. Growth hormone and thyroid-stimulating hormone secretion are regulated by somatostatin receptor subtypes SSTR2 and SSTR5, with an additional effect on growth hormone secretion via SSTR1. Activation of somatostatin receptor types SSTR2 and SSTR5 have been associated with growth hormone suppression and more particularly growth hormone secreting adenomas (acromegaly) and thyroid-stimulating hormone secreting adenomas. Prolactin is regulated by SSTR5 alone.

The clinically available somatostatin analogues, octreotide (Sandostatin®) and lanreotide, are used for the treatment of acromegaly patients for whom surgery has failed to adequately control growth and insulin-like growth factor I (IGF-I) levels or where surgery is contra-indicated. Both analogues exhibit selective high affinity for somatostatin receptor subtype 2 (SSTR2). Sandostatin® binds mainly to SSTR2 and to some extent to the SSTR3 and SSTR5.

Pasireotide was developed for the approved Sandostatin® indications, but as a more potent somatostatin analogue with a longer plasma half-life in vivo. Lewis I et al., J Med Chem 46(12): 2334-44 (Jun. 5, 2003); Weckbecker G et al., Endocrinology 143(10): 4123-30 (October 2002). In contrast with other analogues, pasireotide binds to all somatostatin receptors except SSTR4. The binding affinity for the different somatostatin receptors was a basis for defining the scope of possible new clinical indications for pasireotide. Bruns C et al., Eur J Endocrinol 143(Suppl 1): S3-7 (2000); Bruns C et al., Eur J Endocrinol. 146(5):707-16 (May 2002). In addition, other possible new indications were suggested due to the improved activity of pasireotide for growth hormone and IGF-1 regulation and its different inhibitory effects on insulin and glucagon secretions.

A somatostatin analogue with universal high affinity somatostatin binding, such as pasireotide, will not only have greater efficacy for growth hormone inhibition, but will also regulate secretion of additional anterior pituitary hormones. Murray R D et al., Endocrine Abstracts 5: P186 (2003). A clear signature for pasireotide, even at sub-therapeutic dose, could identify the somatostatin agonist activity consistent with the known pharmacological action of the pasireotide class of compounds. This signature would be potentially usable to compare the activity in different tissues treated with somatostatin or somatostatin analogues.

Accordingly, there is a need in the art for an organism-wide understanding of the activity of somatostatin analogues.

### SUMMARY OF THE INVENTION

The invention also provides a method for treating a condition in a subject, wherein the condition is one for which administration of somatostatin or a somatostatin analogue is indicated. The method involves, first administering a compound of interest to the subject (e.g., a primate subject) and then obtaining the gene expression profile of the subject following administration of the compound. The gene expression profile of the subject is compared to a biomarker gene expression profile. The biomarker gene expression profile is indicative of efficacy of treatment by somatostatin or a somatostatin analogue. In one embodiment, the biomarker gene expression profile is the baseline gene expression profile of the subject before administration of the compound. In another embodiment, the biomarker gene expression profile is the gene expression profile or average of gene expression profiles of a vertebrate to whom somatostatin or a somatostatin analogue (e.g. pasireotide) has been administered. A similarity in the gene expression profile of the subject to whom the compound was administered to the biomarker gene expression profile is indicative of efficacy of treatment with the compound.

The invention provides biological markers of somatostatin or somatostatin analogue efficacy. The effects on the growth hormone/IGF-1 and glucagon/insulin axes were reflected in transcript level changes in several organs. The expressed genes are useful as surrogate markers of the biological activity of pasireotide, especially the findings for IGF-2 in the pituitary and kidneys. The biomarker signature can be used to compare treatment efficacy in different tissues in an organism treated with somatostatin or somatostatin analogues.

The invention provides methods for determining a subject for inclusion in a clinical trial, based upon an analysis of biomarkers expressed in the subject to be treated. The compound to be tested is administered to the subject. In one embodiment, the compound to be tested is administered in a sub-therapeutic dose. For example, a clear signature for pasireotide, even at sub-therapeutic dose, could identify the somatostatin agonist activity consistent with the known pharmacological action of the pasireotide class of compounds. This signature would be potentially usable to compare the activity in different tissues treated with somatostatin or somatostatin analogues. Then, the gene expression profile of the subject following administration of the compound is obtained. The subject may be included in the clinical trial when the gene expression profile of the subject to whom the compound was administered is similar to a biomarker gene expression profile indicative of efficacy of treatment by somatostatin or a somatostatin analogue. The subject may be excluded from the clinical trial when the gene expression profile of the subject is dissimilar to the biomarker gene expression profile indicative of efficacy of treatment. Such similarities or dissimilarities are observable to those of skill in the art.

The invention also provides for the use of pasireotide in the manufacture of a medicament for the treatment of disorders of growth regulation in a selected patient population. The patient population is selected on the basis of a gene expression profile indicative of pasireotide efficacy by the patient to whom pasireotide is administered.

The invention also provides a method for determining whether a compound has a therapeutic efficacy similar to that of somatostatin or a somatostatin analogue, such as pasireotide. The compound is administered to the subject, and then a gene expression profile of the subject as a consequence of administration of the compound is obtained. The resulting gene expression profile of the subject is compared to a standard biomarker gene expression profile indicative of efficacy of treatment by somatostatin or a somatostatin analogue. The compound is determined to have therapeutic efficacy similar to that of somatostatin or a somatostatin analogue when the gene expression profile of the subject is similar to a standard biomarker gene expression profile, but the compound is determined to have therapeutic efficacy different from that of somatostatin or a somatostatin analogue when the gene expression profile of the subject is different from a standard biomarker gene expression profile.

The invention also provides clinical assays, kits and reagents for determining treatment efficacy of a condition for which administration of somatostatin or a somatostatin analogue is indicated. In one embodiment, the kits contain reagents for determining the gene expression of biomarker genes, by hybridization. In another embodiment, the kits contain reagents for determining the gene expression of biomarker genes, by the polymerase chain reaction.

### DETAILED DESCRIPTION OF THE INVENTION

The invention provides for the identification of the mode of action and potential therapeutic indication of somatostatin or somatostatin analogues by multiorgan microarray analysis, e.g. in cynomolgus monkeys. The invention provides for the assessment as to what extent the transcriptional profiles of the various tissues could be used for a comparison of the pharmacological profile of pasireotide with somatostatin, Sandostatin), or other somatostatin analogues.

As used herein, a gene expression profile is diagnostic for determining the efficacy of treatment when the increased or decreased gene expression is an increase or decrease (e.g., at least a 1.5-fold difference) over the baseline gene expression following administration of the compound. Alternatively or in addition, the gene expression profile is diagnostic for determining the efficacy of treatment as compared with treatment of somatostatin or somatostatin analogues (e.g., pasireotide) when the gene expression profile of the treated subject is comparable to a standard biomarker gene expression profile. In one embodiment, the standard biomarker gene expression profile is the gene expression profile or average of gene expression profiles of a vertebrate to whom somatostatin or a somatostatin analogue has been administered, this profile or profile being the standard to which the results from the subject following administration is compared. Such an approach, which contains aspects of therapeutics and diagnostics, is termed “theranostic” by many of those of skill in the art.

In one embodiment, the subject is a vertebrate. In a particular embodiment, the vertebrate is a mammal. In a more particular embodiment, the mammal is a primate, such as a cynomolgus monkey or a human. As used herein, the administration of an agent or drug to a subject or patient includes self-administration and the administration by another.

As used herein, a gene expression pattern is “higher than normal” when the gene expression (e.g., in a sample from a treated subject) shows a 1.5-fold difference (i.e., higher) in the level of expression compared to the baseline samples. A gene expression pattern is “lower than normal” when the gene expression (e.g., in a sample from a treated subject) shows a 1.5-fold difference (i.e., lower) in the level of expression compared to the baseline samples.

Techniques for the detection of gene expression of the genes described by this invention include, but are not limited to northern blots, RT-PCT, real time PCR, primer extension, RNase protection, RNA expression profiling and related techniques. Techniques for the detection of gene expression by detection of the protein products encoded by the genes described by this invention include, but are not limited to, antibodies recognizing the protein products, western blots, immunofluorescence, immunoprecipitation, ELISAs and related techniques. These techniques are well known to those of skill in the art. Sambrook J et al., Molecular Cloning: A Laboratory Manual, Third Edition (Cold Spring Harbor Press, Cold Spring Harbor, 2000). In one embodiment, the technique for detecting gene expression includes the use of a gene chip. The construction and use of gene chips are well known in the art. See, U.S. Pat. Nos. 5,202,231; 5,445,934; 5,525,464; 5,695,940; 5,744,305; 5,795,716 and 5,800,992. See also, Johnston, M. Curr Biol 8:R171-174 (1998); Iyer V R et al., Science 283:83-87 (1999) and Elias P, “New human genome ‘chip’ is a revolution in the offing” Los Angeles Daily News (Oct. 3, 2003).

Somatostatin and somatostatin analogues. The peptides and therapeutic uses of somatostatin-14 and somatostatin-28 are well known in the art. See, U.S. Pat. No. 6,225,284; Lewis I et al., J. Med. Chem. 46(12): 2334-44 (Jun. 5, 2003); Weckbecker G et al., Endocrinology 143(10): 4123-30 (October 2002), each incorporated herein by reference. Somatostatin and somatostatin analogues in free form or in the form of pharmaceutically acceptable salts and complexes exhibit valuable pharmacological properties as indicated in in vitro and in vivo tests and are therefore indicated for therapy.

By “somatostatin analogue” as used herein is meant a straight-chain or cyclic peptide derived from that of the naturally occurring somatostatin-14, wherein one or more amino acid units have been omitted or replaced by one or more other amino acid radicals or wherein one or more functional groups have been replaced by one or more other functional groups and/or one or more groups have been replaced by one or several other isosteric groups. See, U.S. Pat. No. 6,225,284, incorporated herein by reference. Cyclic, bridge cyclic and straight-chain somatostatin analogues are known compounds. Such compounds and their preparation are described e.g. in European Patent Specifications EP-A-1295; 29,579; 215,171; 203,031; 214,872; 298,732; 277,419. In general the term “somatostatin analogue” covers all modified derivatives of the native somatostatin-14 that have binding affinity in the nM range to at least one somatostatin receptor subtype.

One somatostatin analogue of interest is pasireotide, which has a chemical structure cyclo[4-(NH₂—C₂H₄—NH—CO—O)Pro-Phg-DTrp-Lys-Tyr(4-Bzl)-Phe] as follows:

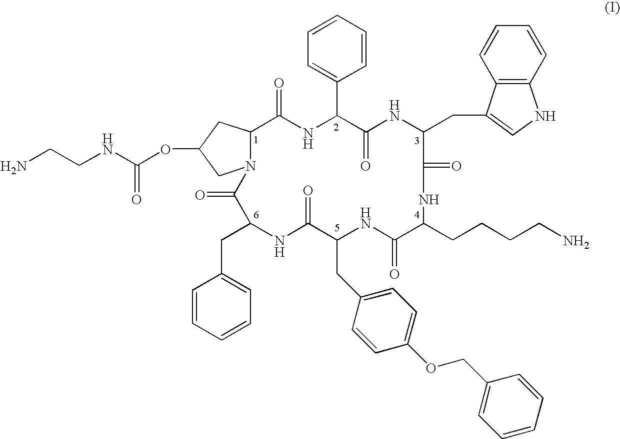

Here, Phg means —HN—CH(C₆H₅)—CO— and Bzl means benzyl. See, PCT patent application WO 02/10192. Pasireotide is a somatostatin analogue with binding affinities for the five somatostatin receptors except somatostatin receptor 4 (SSTR4). Pasireotide has been developed for several indications, including those disclosed above for other somatostatin analogues. See, Lewis I et al., J. Med. Chem. 46(12):2334-44 (Jun. 5, 2003); Weckbecker G et al., Endocrinology 143(10): 4123-4130 (2002); Kneissel M et al., Bone 28:237-250 (2001); and Thomsen J S et al., Bone 25:561-569 (1999), the contents of which are incorporated herein by reference.

Somatostatins and somatostatin analogues bind to somatostatin receptors (SSTR). The cellular effects of somatostatin receptor activation are currently understood to be as follows: Binding to somatostatin receptors results in the activation of the PI3 kinase signalling pathway, inhibition of adenylyl cyclase, activation of protein tyrosine phosphatases, modulation of mitogen activated protein kinase (MAPK), coupling to inward rectifying K⁺ channels, voltage dependent Ca⁺⁺ channels, a Na⁺/H⁺ exchanger, AMPA/kainate glutamate channels, PLC, and PLA2. Patel Y C, Frontiers in Neuroendocrinology 20: 157-98 (1999). Somatostatin receptor activation blocks cell secretion by inhibiting intracellular cAMP and Ca⁺⁺ and by a receptor-linked distal effect on exocytosis. Somatostatin receptor 1, 2, 4 and 5 (SSTR1, 2, 4, 5) induce cell cycle arrest by phosphotyrosine phosphatase-dependent modulation of MAPK, associated with induction of the retinoblastoma (Rb) tumour suppressor protein and p21. SSTR3 triggers phosphotyrosine phosphatase-dependent apoptosis accompanied by activation of p53 and Bax.

Additional effects of treatment of primates with somatostatin, in particular with the somatostatin analogue pasireotide, are provided in the EXAMPLE below.

Somatostatin and somatostatin analogues bind to at least one somatostatin receptor subtype. Five somatostatin receptor subtypes, SST-1, SST-2, SST-3, SST-4 and SST-5 have been cloned and characterized. Human somatostatin receptors hSST-1, hSST-2 and hSST-3 and their sequences have been disclosed by Yamada Y et al., Proc. Nat. Acad. Sci. U.S.A. 89: 251-255 (1992). Human somatostatin receptor hSST-4 and its sequence have been disclosed by Rohrer L et al., Proc. Acad. Sci. U.S.A. 90: 4196-4200 (1993). Human somatostatin receptor hSST-5 and its sequence have been described by Panetta R et al., Mol. Pharmacol. 45: 417-427 (1993).

Binding assays maybe carried out using membranes prepared from hSST-1, hSST-2, hSST-3, hSST-4 or hSST-5 selective cell lines, e.g. CHO cells stably expressing hSST-1, hSST-2, hSST-3, hSST4 or hSST-5. See, U.S. Pat. No. 6,225,284. Somatostatin and somatostatin analogues have in the above binding assays towards hSST-1, hSST-2, hSST-3, hSST-4 and/or hSST-5 an IC₅₀ in the nM range.

Furthermore, somatostatin and somatostatin analogues show growth hormone-release inhibiting activity as indicated by the inhibition of GH release in vitro from cultured pituitary cells. See, U.S. Pat. No. 6,225,284. Somatostatin and somatostatin analogues inhibit the release of growth hormone concentration-dependent from 10⁻¹¹ to 10⁻⁶ M.

Somatostatin and somatostatin analogues also inhibit the release of insulin and/or glucagon, as indicated in standard tests using male rats. See, U.S. Pat. No. 6,225,284. The determination of the blood serum insulin and glucagon levels is effected by radioimmunoassay. Somatostatin and somatostatin analogues are active in this test when administered at a dosage in the range of from 0.02 to 1000 μg/kg subcutaneous (s.c.), e.g. to 10 μg/kg s.c.

As described above, however, the administration of somatostatin or somatostatin analogues, even in sub-therapeutic doses, can usefully provide biomarker signature information.

Somatostatin and somatostatin analogues are useful for the treatment of disorders with an aetiology comprising or associated with excess growth-secretion, e.g. in the treatment of acromegaly as well as in the treatment of diabetes mellitus, especially complications thereof (e.g. angiopathy, proliferative retinopathy, dawn phenomenon and nephropathy and other metabolic disorders related to insulin or glucagon release). See, U.S. Pat. No. 6,225,284. Somatostatin and somatostatin analogues also inhibit gastric acid secretion, exocrine and endocrine pancreatic secretion and the secretion of various peptides of the gastrointestinal tract. Somatostatin and somatostatin analogues additionally are useful for the treatment of gastrointestinal disorders, for example in the treatment of peptic ulcers, enterocutaneous and pancreaticocutaneous fistula, irritable bowel syndrome and disease, dumping syndrome, watery diarrhoea syndrome, ADDS-related diarrhoea, chemotherapy-induced diarrhoea, acute or chronic pancreatitis and gastrointestinal hormone secreting tumours (e.g. vipomas, glucagonomas, insulinomas, carcinoids and the like) as well as gastrointestinal bleeding. Somatostatin and somatostatin analogues are also effective in the treatment of tumours which are somatostatin receptor positive, particularly tumours bearing human somatostatin receptors hSST-1, hSST-2, hSST-3, hSST-4 and/or hSST-5. Somatostatin and somatostatin analogues are useful for treating an aetiology comprising or associated with excess growth hormone-secretion, for treating gastrointestinal disorders, for inhibiting proliferation or keratinisation of epidermal cells, or for treating degenerative senile dementia in a subject in need of such a treatment. See, U.S. Pat. No. 6,123,916, incorporated herein by reference. Somatostatin and somatostatin analogues are also useful for treating tuberculosis, sarcoidosis, malignant lymphoma, Merkel cell tumour of the skin, osteosarcoma, focal lymphocytic reaction, localized autoimmune disease, and organ rejection after transplantation. See, U.S. Pat. No. 6,123,916. Somatostatin and somatostatin analogues are particularly indicated for the treatment of somatostatin receptor positive tumours, e.g. cancers of the breast, prostate, colon, pancreas, brain, lung and lymph nodes.

To reiterate, somatostatin and somatostatin analogues have been developed and are being used to treat several indications, including acromegaly, diabetes mellitus and complications (e.g. angiopathy, diabetic proliferative retinopathy, diabetic macular oedema, nephropathy, neuropathy, hypothalamic or hyperinsulinaemic obesity), morbid obesity, Grave's Disease, polycystic kidney disease gastrointestinal disorders (e.g. irritable bowel syndrome and disease or enterocutaneous and pancreaticocutaneous fistula), dumping syndrome, watery diarrhoea syndrome, AIDS-related diarrhoea, chemotherapy-induced diarrhoea, pancreatitis, gastrointestinal hormone secreting tumours (e.g. GEP tumours, for example vipomas, glucagonomas, insulinomas, carcinoids and the like), somatostatin receptor positive tumours (e.g. pituitary, gastroenteropancreatic, carcinoids, central nervous system, breast, prostatic (including advanced hormone-refractory prostate cancer), ovarian or colonic tumours, small cell lung cancer, malignant bowel obstruction, paragangliomas, kidney cancer, skin cancer, neuroblastomas, pheochromocytomas, medullary thyroid carcinomas, myelomas, lymphomas, Hodgkins and non Hodgkins lymphomas, bone tumours and metastases, chronic allograft rejection and other vascular occlusive disorders (e.g. vein graft stenosis, restenosis and/or vascular occlusion following vascular injury, e.g. caused by cauterisation procedures or vascular scraping procedures such as percutaneous transluminal angioplasty, laser treatment or other invasive procedures which disrupt the integrity of the vascular intima or endothelium), angiogenesis, hepatocellular carcinoma as well as gastrointestinal bleeding (e.g. variceal oesophageal bleeding), macular oedema (e.g. cystoid macular oedema, idiopathic cystoid macular oedema, exudative age-related macular degeneration, choroidal neovascularisation related disorders) and proliferative retinopathy. Somatostatin and somatostatin analogues are used for treating Cushing disease, a subtype of pituitary tumours. Somatostatin and somatostatin analogues are also used for treating sleep apnoea.

Somatostatin and somatostatin analogues, either free or in complexed form, may be administered by any conventional route, in particular intraperitoneally or intravenously, e.g. in the form of injectable solutions or suspensions. They may also be administered advantageously by infusion, e.g. an infusion of 30 to 60 min. Depending on the site of the tumour, they may be administered as close as possible to the tumour site, e.g. by means of a catheter. A pharmaceutical composition comprising somatostatin or somatostatin analogues in free or complexed form together with one or more pharmaceutically acceptable carriers or diluents may be manufactured in conventional manner and may be presented, e.g. for imaging, in the form of a kit. See, U.S. Pat. No. 6,225,284.

Somatostatin and somatostatin analogues can be administered in combination with other drugs, such as Starlix® or other anti-diabetic drugs, or a chemotherapeutic agent, e.g. paclitaxel, gemcitabine, doxorubicin, 5-fluorouracil, taxol, an anti-androgen, mitoxanthrone, antioestrogen, e.g. letrozole, an antimetabolite, a plant alkaloid, a lymphokine, interferons, an inhibitor of protein tyrosine kinase and/or serine/threonine kinase, epothilone, or an anti-angiogenic agent.

The kits of the invention may contain a written product on or in the kit container. The written product describes how to use the reagents contained in the kit to determine whether a patient is being treated with a compound for which treatment by somatostatin or a somatostatin analogue is indicated. In several embodiments, the use of the reagents can be according to the methods of the invention. In one embodiment, the reagent is a gene chip for determining the gene expression of relevant genes.

The following EXAMPLE is presented in order to more fully illustrate the preferred embodiments of the invention. This EXAMPLE should in no way be construed as limiting the scope of the invention, as defined by the appended claims.

### EXAMPLE

Pasireotide-Induced Gene Expression Profiling in Monkeys

Introduction and summary. Microarray gene expression assays were performed using tissues of monkeys treated with pasireotide at sub-therapeutic dose for 14 days. The assays were analyzed to identify the modes of actions of pasireotide with relationships to therapeutic applications.

All monkey tissues examined (thyroid, brown fat, pituitary, pancreas, liver, kidney, spleen) demonstrated changes in the genes regulated by the binding of the natural somatostatin 14 (SST-14) and somatostatin 28 (SST-28) to somatostatin receptors (SSTRs). The transcript profiles reflected the known somatostatin actions on the growth hormone/insulin-like growth factor 1 (GH/IGF-1), glucagon/insulin axes and on cell proliferation. However, the compound affected significantly the transcript levels of other related genes like insulin-like growth factor 2 (IGF-2) in the pituitary and kidneys. This could be a candidate biological marker (biomarker) of drug efficacy provided that the change in protein biosynthesis would be reflected in an easily accessible tissue like the blood. Other known effects of somatostatin and agonists on growth factors, cells of the immune system and the cardio-vascular and renal functions were also reflected by the changes in the profiles of these classes of genes after pasireotide.

Origin of tissue and processing. Male and female cynomolgus monkeys received subcutaneously pasireotide (100 μg/animal/day) or the vehicle for 14 days. On day 15, all animals were sacrificed and tissues for RNA extraction were immediately snap-frozen and kept at −80° C. until processing.

TABLE 1

| | | | | | |
|---|---|---|---|---|---|
| Tissue | Animal or | | | | Dose |
| Sample | sample no. | Sex | Tissue/organ | Compound | (μg/animal/day) |

Origin of Tissues Used for Analysis

| | | | | | |
|---|---|---|---|---|---|
| x547e | W62405 | Male | Brown fat | Pasireotide | 100 |
| x548e | W62406 | Male | Brown fat | Pasireotide | 100 |
| x549e | W62425 | Female | Brown fat | Pasireotide | 100 |
| x550e | W62426 | Female | Brown fat | Pasireotide | 100 |
| x673e | W62401 | Male | Brown fat | Control | 0 |
| x675e | W62421 | Female | Brown fat | Control | 0 |
| x676e | W62422 | Female | Brown fat | Control | 0 |
| x857e | W62501* | Male | Brown fat | Control | 0 |
| x858e | W62502* | Male | Brown fat | Control | 0 |
| x859e | W62551* | Female | Brown fat | Control | 0 |
| x860e | W62552* | Female | Brown fat | Control | 0 |
| d32e | W62551 | Female | Kidney | Control | 0 |
| d35e | W62502 | Male | Kidney | Control | 0 |
| d37e | W62552 | Female | Kidney | Control | 0 |
| d45e | W62501 | Male | Kidney | Control | 0 |
| x407e | W62401 | Male | Kidney | Control | 0 |
| x408e | W62402 | Male | Kidney | Control | 0 |
| x409e | W62421 | Female | Kidney | Control | 0 |
| x410e | W62422 | Female | Kidney | Control | 0 |
| x521e | W62405 | Male | Kidney | Pasireotide | 100 |
| x522e | W62406 | Male | Kidney | Pasireotide | 100 |
| x523e | W62425 | Female | Kidney | Pasireotide | 100 |
| x524e | W62426 | Female | Kidney | Pasireotide | 100 |
| x401e | W62401 | Male | Liver left lateral lobe | Control | 0 |
| x402e | W62402 | Male | Liver left lateral lobe | Control | 0 |
| x403e | W62421 | Female | Liver left lateral lobe | Control | 0 |
| x404e | W62422 | Female | Liver left lateral lobe | Control | 0 |
| x517e | W62405 | Male | Liver left lateral lobe | Pasireotide | 100 |
| x518e | W62406 | Male | Liver left lateral lobe | Pasireotide | 100 |
| x519e | W62425 | Female | Liver left lateral lobe | Pasireotide | 100 |
| x520e | W62426 | Female | Liver left lateral lobe | Pasireotide | 100 |
| x529e | W62405 | Male | Pancreas | Pasireotide | 100 |
| x530e | W62406 | Male | Pancreas | Pasireotide | 100 |
| x531e | W62425 | Female | Pancreas | Pasireotide | 100 |
| x532-2e | W62426 | Female | Pancreas | Pasireotide | 100 |
| x641e | W62401 | Male | Pancreas | Control | 0 |
| x642e | W62402 | Male | Pancreas | Control | 0 |
| x645e | W62421 | Female | Pancreas | Control | 0 |
| x646e | W62422 | Female | Pancreas | Control | 0 |

Origin of Tissues

| | | | | | |
|---|---|---|---|---|---|
| x413e | W62401 | Male | Pituitary gland | Control | 0 |
| x414e | W62402 | Male | Pituitary gland | Control | 0 |
| x415e | W62421 | Female | Pituitary gland | Control | 0 |
| x513-2e | W62405 | Male | Pituitary gland | Pasireotide | 100 |
| x514e | W62406 | Male | Pituitary gland | Pasireotide | 100 |
| x515e | W62425 | Female | Pituitary gland | Pasireotide | 100 |
| x516e | W62426 | Female | Pituitary gland | Pasireotide | 100 |
| x425e | W62401 | Male | Spleen | Control | 0 |
| x426e | W62402 | Male | Spleen | Control | 0 |
| x427e | W62421 | Female | Spleen | Control | 0 |
| x428e | W62422 | Female | Spleen | Control | 0 |
| x525e | W62405 | Male | Spleen | Pasireotide | 100 |
| x526e | W62406 | Male | Spleen | Pasireotide | 100 |
| x527e | W62425 | Female | Spleen | Pasireotide | 100 |
| x528e | W62426 | Female | Spleen | Pasireotide | 100 |
| d33e | W62501 | Male | Thyroid | Control | 0 |
| d40e | W62551 | Female | Thyroid | Control | 0 |
| d43e | W62502 | Male | Thyroid | Control | 0 |
| d48e | W62552 | Female | Thyroid | Control | 0 |
| x443e | W62401 | Male | Thyroid | Control | 0 |
| x445e | W62421 | Female | Thyroid | Control | 0 |
| x446e | W62422 | Female | Thyroid | Control | 0 |
| x505e | W62425 | Female | Thyroid | Pasireotide | 100 |
| x506e | W62426 | Female | Thyroid | Pasireotide | 100 |
| x507e | W62405 | Male | Thyroid | Pasireotide | 100 |
| x508e | W62406 | Male | Thyroid | Pasireotide | 100 |

RNA expression profiling was conducted by means of the HG-U95A gene expression probe array (Affymetrix; Santa Clara, Calif. USA), containing more than 12,600 probe sets interrogating primarily full-length human genes and also some control probe sets. The experiment was conducted according to the recommendations of the manufacturer. Briefly, total RNA was obtained by acid guanidinium thiocyanate-phenol-chloroform extraction (Trizol®, Invitrogen Life Technologies, San Diego, Calif. USA) from each frozen tissue section. The total RNA was then purified on an affinity resin (Rneasy®, Qiagen) and quantified. Double stranded cDNA was synthesized with a starting amount of approximately 5 μg full-length total RNA using the Superscript® Choice System (Invitrogen Life Technologies, Carlsbad, Calif. USA) in the presence of a T7-(dT)24 DNA oligonucleotide primer. Following synthesis, the cDNA was purified by phenol/chloroform/isoamyl alcohol extraction and ethanol precipitation. The purified cDNA was then transcribed in vitro using the BioArray® High Yield RNA Transcript Labeling Kit (ENZO, Farmingdale, N.Y. USA) in the presence of biotinylated ribonucleotides form biotin labelled cRNA. The labelled cRNA was then purified on an affinity resin (Rneasy®, Qiagen), quantified and fragmented. An amount of approximately 10 μg labelled cRNA was hybridized for 16 hours at 45° C. to an expression probe array. The array was then washed and stained twice with streptavidin-phycoerythrin (Molecular Probes,) using the GeneChip® Fluidics Workstation 400 (Affymetrix, Santa Clara, Calif. USA). The array was then scanned twice using a confocal laser scanner (GeneArray® Scanner, Agilent, Palo Alto, Calif. USA) resulting in one scanned image. This resulting “.dat-file” was processed using the MAS4 program (Affymetrix) into a “.cel-file”. The “.cel file” was captured and loaded into the Affymetrix GeneChip® Laboratory Information Management System (LIMS). The LIMS database is connected to a UNIX Sun Solaris server through a network filing system that allows for the average intensities for all probes cells (CEL file) to be downloaded into an Oracle database (NPGN). Raw data was converted to expression levels using a “target intensity” of 150. The data were evaluated for quality control and loaded in the GeneSpring® software 4.2.4 (Silicon Genetics, Calif. USA) for analysis.

On the human Affymetrix HGU95Av2 chip, probe sets for individual genes contain 20 oligonucleotide pairs, each composed of a “perfect match” 25-mer and a “mismatch” 25-mer differing from the “perfect” match oligonucleotide at a single base. After probe labelling, hybridization, and laser scanning, the expression level was estimated by averaging the differences in signal intensity measured by oligonucleotide pairs of a given probe (AvgDiff value). The fold changes and directions were calculated for selected genes, from the differences of the AvgDiff values between controls and treated.

To identify genes that were impacted by pasireotide, the dataset was initially filtered to exclude in a first wave of analysis, genes whose values were systematically in the lower expression ranges where the experimental noise is high (at least 80 in a number of experiments corresponding to the smallest number of replicas of any experimental point). In a second round of selection a threshold p-value of 0.05 (based on a t-test) identified differences between treated and control based on a two component error model (Global Error Model) and, whenever possible, with a stepdown correction for multi-hypothesis testing (Benjamini and Hochberg false discovery rate). The decision to keep or reject a specific gene was based on the conjunction of numerical changes identified by comparative and statistical algorithms and the relationship to other modulated genes that point to a common biological theme. The weight of this relationship was assessed by the analyst through a review of the relevant scientific literature.

For the assay analysis described herein: (1) the increase and decrease in expression referred to the RNA expression level unless specifically stated; (2) if there were multiple probe sets representing the same gene, the probe set designed for sense target was favoured; and (3) the changes in gene expression indicated that a pathway, a cellular activity or component represented by an individual gene might be impacted. Understanding the functional implication is dependent on the information available on the biological context of the transcript level change (gene function, physiological variation, other gene changes, tissue, compound, etc.). RT-PCR is used to identify the extent of absolute change in mRNA levels, but this method in general does not add more information on the relevance of the transcript level changes.

Among tie 12,600 genes per chip, about 100 genes were found to reflect the compound signature in a particular tissue. For clarity, they were divided in different classes and subdivided, with many overlaps, into functional categories in the following TABLE.

TABLE 2
Pasireotide Gene Expression Profiling

| CLASS | PITUITARY | BROWN FAT | PANCREAS |
|---|---|---|---|
| SIGNAL | | | |
| TRANSDUCTION | | | |
| 1) Phophatidyl inositol | IP-4-phosphatase, | PI-3-kinase. regulatory | IP-1-phosphatase |
| and related | type 1, isoform b ↓ x2 | subunit, polypeptide 2 | ↑ x2.5 |
| pathways/PKC, | PI-3-kinase, catalytic, | (p85 β) ↓ x3.5 | PI-4-kinase, catalytic, |
| phospholipases | α polypeptide ↓ x3 | PI glycan, class F ↓ x2 | α polypeptide ↑ x1.5 |
| | PI-3-kinase, catalytic, | PLC β 4 ↑ x2 | PL A2, group IVC |
| | δ polypeptide | PI glycan, class L ↑ | (cytosolic, calcium- |
| | ↓ x2 | x3.5 | independent) ↑ x1.5 |
| | 1-PI-4-phosphate 5- | | |
| | kinase isoform C ↓ x1.5 | | |
| | PI transfer protein, β | | |
| | ↓ x2.5 | | |
| | PLCγ 1 ↓ x1.5 | | |
| | PKC inhibitor ↑ x2 | | |
| | IP3 receptor, type 1 | | |
| | ↑ x1.5 | | |
| 2) Other calcium/ | Calcium/calmodulin- | Calcium/calmodulin- | |
| calcineurin/calmodulin | dependent protein | dependent protein | |
| dependent pathways | kinase I ↑ x2.5 | kinase I ↓ x3.5 | |
| and associated proteins | Receptor (calcitonin) | | |
| | activity modifying | | |
| | protein 2 precursor ↑ | | |
| | x3.5 | | |
| 3) Ras/MAPK | Rab geranylgeranyl | Ras homolog gene | SH3 domain |
| kinase/ERK kinase | transferase, α subunit | family, member G | binding glutamic |
| related pathways and | ↓ x1.5 | (rho G) | acid-rich protein |
| adaptor proteins | Rab3 GTPase- | MAPKAPK 3 | MAPKAPK 3 |
| | activating protein, non | MAPKK1 | Ras like GTPase |
| | catalytic subunit ↓ x2 | MAPK 8 | RaP2 interacting |
| | SHB adaptor protein | RAB6, member RAS | protein 8 |
| | (a Src homology 2 | oncogene family | |
| | protein) | Adaptor-related | |
| | ↓ x2.5 | protein complex 3, σ 1 | |
| | MAPKKK5 ↑ x2 | subunit | |
| | Rab | Ras-related nuclear | |
| | geranyltransferase, | protein | |
| | β subunit ↑ x2 | Rab acceptor 1 | |
| | RAB 5C, member | (prenylated) | |
| | RAS oncogene family | RAB 2, member RAS | |
| | ↑ x3 | oncogene family | |
| | | IQ motif containing | |
| | | GTPase activating | |
| | | protein 2 | |
| 4) JAK/STAT pathway | STAT 1, 91kδ ↓ x2 | JAK 3 | STAT 5B |
| and related kinases | JAK 1 ↑ x2 | | STAT 1, 91kδ |
| | | | STAT 2 |
| 5) Protein tyrosine | Dual specificity | PP 2, regulatory | PTP δ |
| phosphatases/other | phosphatase 8 ↓ x3 | subunit B (B56), γ | PP 1, regulatory |
| phosphatases | Phosphatase and | isoform ↓ x1.5 | (inhibitor) subunit 8 |
| | tensin homolog | PP 5, catalytic subunit | PP 2A, catalytic |
| | (mutated in multiple | ↑ x2.5 | subunit B′ |
| | advanced cancers 1) | Dual specificity PP | PP 1A (formerly |
| | ↓ x3.5 | MKP-5 ↑ x2.5 | 2C), magnesium- |
| | PTP, receptor type, T | | dependent, α |
| | ↑ x2 | | isoform |
| | PP 1, regulatory | | PP 2A, regulatory |
| | (inhibitor) subunit 5 | | subunit B′ |
| | ↑ x3.5 | | Dual specificity |
| | | | phosphatase 8 |
| 6) Other protein kinases | Arg PTK-binding | PTK 9-like (A6-related | Protein kinase (cAMP- |
| and associated binding | protein ↓ x2.5 | protein) | dependent, |
| proteins | | PTK A kinase (PRKA) | catalytic), inhibitor γ |
| | | anchor protein 1 | Serine/threonine |
| | | cAMP-dependent | protein kinase |
| | | protein kinase R1-β | Receptor PTK |
| | | regulatory subunit | Serine/threonine |
| | | Ribosomal protein S6 | kinase 11 (Peutz- |
| | | kinase, 90 kD, | Jeghers syndrome) |
| | | polypeptide | Tyrosine kinase |
| | | | Ribosomal |
| | | | protein S6 kinase, |
| | | | 90kδ, polypeptide 3 |
| 7) Adenylate/guanylate | Soluble adenylyl | | |
| cyclases and related | cyclase ↓ x2 | | |
| pathways | | | |
| CELL SURFACE | | | |
| RECEPTORS | | | |
| 1) G-protein coupled | GTP-binding protein | G α inhibiting activity | G protein-coupled |
| receptors and related | (G protein), q | polypeptide 3 interacting | receptor 39 |
| binding proteins/ | polypeptide | protein ↓ x5 | G protein-coupled |
| G proteins | ↓ x2.5 | G protein-coupled | receptor 49 |
| | GTP-binding protein | receptor 1 ↓ x2.5 | G protein-coupled |
| | like-1 ↓ x3 | Guanine nucleotide | receptor 3 |
| | G-protein coupled | binding protein (G | Regulator of G- |
| | receptor 49 ↓ x2 | protein), β polypeptide 3 | protein signalling 10 |
| | G protein-coupled | ↑ x2.5 | GTP-binding |
| | receptor, family C, | SSTR3↑ x6.5 | protein |
| | group 5, member B ↑ x2 | Endothelial | SSTR2 ↓ x1.5 |
| | GTP-binding protein | differentiation, | |
| | 11 ↑ x2.5 | sphingolipid G-protein- | |
| | Receptor tyrosine | coupled receptor, 5 | |
| | kinase-like orphan | ↑ x2.5 | |
| | receptor 2 ↑ x2 | | |
| | ATP(GTP)-binding | | |
| | protein ↑ x1.5 | | |
| | G-protein coupled | | |
| | receptor 9 ↑ x2.5 | | |
| | Regulator of G-protein | | |
| | signalling 9 | | |
| | ↑ x2 | | |
| | SSTR3 ↑ x3 | | |
| 2) Growth factors, their | FGFR 2 ↓ x2 | Fms-related tyrosine | Smad 3 ↓ x1.5 |
| receptors and related | EGFRBP 2 ↓ x1.5 | kinase 1 (VEGF/ | G-CSF protein |
| binding proteins | Fms-related tyrosine | vascular permeability | ↓ x2 |
| | kinase 1 | factor receptor) ↓ x4.5 | PDGFR α ↑ x2.5 |
| | (VEGF/vascular | EGF receptor pathway | PDGFR, |
| | permeability factor | substrate 15 ↓ x2 | α polypeptide |
| | receptor) ↓ x1.5 | CSF 1 (macrophage) | ↑ x1.5 |
| | Catenin (cadherin- | ↓ x2 | |
| | associated protein), | Cadherin 13, H- | |
| | α 1 (102 kD) ↓ x1.5 | cadherin (heart) ↓ x2 | |
| | PDGFβ ↓ x2 | Cadherin F1B1 ↓ x4 | |
| | GFR bound protein 10 | Endothelial cell GF 1 | |
| | ↑ x1.5 | (platelet derived) ↓ x2 | |
| | Butyrate response | TGF β-activated | |
| | factor 2 (EGF-response | kinase-binding protein 1 | |
| | factor 2) ↑ x3 | ↑ x2.5 | |
| | VEGF B ↑ x1.5 | CSF 3 receptor | |
| | | (granulocyte) ↑ x3 | |
| | | TGF β 3 ↑ x2.5 | |
| | | Cadherin 5, VE- | |
| | | cadherin (vascular | |
| | | epithelium) ↑ x3 | |
| | | VGF nerve growth | |
| | | factor inducible ↑ x2 | |
| | | IL 3 (CSF, multiple) | |
| | | ↑ x2 | |
| | | IL 7R precursor ↑ x2 | |
| 3) Glutamate receptor | GLUR 2, precursor | GLUR, metabotropic 1 | GLUR precursor, |
| and related binding | ↑ x1.5 | ↑ x2 | flip isoform ↑ x3 |
| proteins | | | |
| ATP-DEPENDENT | K+ channel, subfamily | Ca++ channel, voltage | K+ voltage-gated |
| TRANSPORT | K, member 3 (TASK) | dependent, α 1H | channel, KQT-like |
| PROTEINS | ↓ x4 | subunit | subfamily, |
| Ion channels and | K+ voltage-gated | ↑ x3 | member3 |
| related pathways | channel, Shab-related | G protein-activated | ↓ x2.5 |
| | subfamily, member 1 | inwardly rectifying K+ | Ca++ channel, |
| | ↓ x2 | channel ↑ x3.5 | voltage dependent, |
| | ATPase, H+/K+ | | α 1F subunit |
| | exchanging, α | | ↓ x4.5 |
| | polypeptide ↓ x5 | | Na+ channel, |
| | ATPase, Na+/K+ | | voltage-gated, type |
| | transporting, α 2₍₊₎ | | 1, β polypeptide |
| | polypeptide ↑ x2.5 | | ↑ x2 |
| | ATPase, Na+/K+ | | |
| | transporting, β 3 | | |
| | polypeptide ↑ x2.5 | | |
| | ATPase, Ca++ | | |
| | transporting cardiac | | |
| | muscle, slow twitch 2 | | |
| | ↑ x1.5 | | |
| | Putative Ca++ | | |
| | transporting ATPase | | |
| | ↑ x2 | | |
| CELL BIOLOGY/ | | | |
| SPECIALIZED | | | |
| FUNCTIONS | | | |
| 1) Neuromediators/ | Cholinergic receptor, | Dopamine receptor D3 | |
| neuromodulators and | nicotinic, β polypeptide | ↑ x2.5 | |
| related pathways | 4 ↓ x2 | Adrenergic, β-3-, | |
| | Cholinergic receptor, | receptor ↑ x2.5 | |
| | muscarinic 3 ↓ x3 | | |
| | Brain cannabinoid | | |
| | receptor 1 ↑ x2 | | |
| | GABA-B R 1, isoform | | |
| | a precursor | | |
| | ↑ x1.5 | | |
| 2)Pancreatic/gastro- | Cholecystokinin | | Chymotrypsin-like |
| intestinal secretions and | receptor ↓ x4.5 | | ↑ x3.5 |
| related pathways | Gastrin receptor ↓ x2 | | Gastrin-releasing |
| | | | peptide receptor |
| | | | ↓ x5 |
| 3) Hormones and | IGF-2 ↓ x1.5 | THR interactor 10 | CRHR 1 ↓ x2 |
| related pathways | Thyroid transcription | ↓ x3 | THR binding |
| | factor 1 ↑ x2.5 | THR interactor 12 | protein ↓ x2 |
| | Glucagon receptor | ↓ x1.5 | THR interactor 10 |
| | ↑ x7 | IGF-1 ↓ x1.5 | ↑ x2.5 |
| | IGFBP, acid labile | IGF-binding protein 4 | IGF-1 ↑ x4.5 |
| | subunit ↑ x3.5 | ↓ x2 | Prostacyclin |
| | Adrenomedullin ↑ x2.5 | IRS (insulin receptor | synthase ↑ x2 |
| | ANP (atrial natriuretic | substrate) 2 ↑ x2.5 | SSTR2 ↓ 1.5 |
| | peptide precursor B) | T3 receptor ↑ x2 | |
| | ↑ x2 | SSTR3 ↑ 6.5 | |
| | SSTR3 ↑ x3 | Oxytocin, prepro- | |
| | | (neurophysin I) ↑ x2.5 | |
| | | FSHR ↑ x2.5 | |
| 4) Cytoskeleton and | Thrombospondin-p50 | Capping protein (actin | Integrin α 2b |
| associated proteins | ↓ x2 | filament), gelsolin-like | precursor ↑ x1.5 |
| | CD36 antigen ↓ x2 | ↓ x2.5 | |
| | | Actin related protein | |
| | | 2/3 complex, subunit 1A | |
| | | (41 kD) ↓ x2.5 | |
| 5) Enzymes | | Coagulation factor | Thrombospondin 2 |
| | | XIIIAI subunit precursor | ↑ x3.5 |
| | | ↓ x5 | |
| IMMUNITY | TNFR-associated | IFN γ-inducible protein | IL 1 receptor |
| | factor 2 ↓ x4 | 30 (IP30) ↓ x3.5 | antagonist↓ x2 |
| | TNFR subfamily, | Pentaxin-related gene, | LT b4 receptor |
| | member 14; herpesvirus | rapidly induced by IL-1 | (chemokine receptor |
| | entry mediator ↓ x2.5 | ↓ x7.5 | like-1) ↓ x1.5 |
| | IFNR2 (α, β and ω) | IFN induced | Phosphotyrosine |
| | ↓ x2.5 | transmembrane protein | independent ligand |
| | CC chemokines | 1 ↑ x2.5 | p62B for the Lck |
| | STCP-1 ↑ x1.5 | TNF type 1 receptor | SH2 domain B-cell |
| | IFN stimulated gene | associated protein ↓ x2 | isoform ↓ x2 |
| | ↑ x3.5 | IL 5R, α↑ x2.5 | IFN regulatory |
| | IFNγ-inducible protein | CD2 antigen | factor 3 ↑ x5 |
| | 30 (IP30) ↑ x1.5 | (cytoplasmic tail)- | |
| | | binding protein 2 ↑ x2.5 | |
| CELL CYCLE | Forkhead box O3A | G1 to S phase | Cyclin T2 ↓ x2 |
| | ↓ x1.5 | transition ↓ x1.5 | Cyclin D1 ↑ x2 |
| | Cyclin F ↓ x2 | Extra spindle poles, S. cerevisiae, | Cdki 1C ↑ x2 |
| | Core-binding factor, | homologue | |
| | runt domain, α subunit | of ↓ x2.5 | |
| | 2; translocated to, 1; | PCNA ↓ x2.5 | |
| | cyclin D-related ↑ x3 | Follistatin-like 3 | |
| | S-phase response | glycoprotein ↑ x3 | |
| | (cyclin-related) ↑ x2 | Cyclin T2 ↑ x2.5 | |
| | Cell division cycle 25B | | |
| | ↑ x5 | | |
| | Cyclin D3 ↑ x2.5 | | |
| | Cdki2C (p18, inhibits | | |
| | CDK4) ↑ x2 | | |
| | Cdki2D (p19, inhibits | | |
| | CDK4) ↑ x1.5 | | |
| | Forkhead box H1↑ x2 | | |
| APOPTOSIS | BCL2-associated | Neuroblastoma ↓ x2.5 | BCL2/ |
| | athanogene ↑ x2 | Neuroblastoma | adenovirus E1B |
| | BCL2-antagonist of | apoptosis-related RNA | 19 kD-intracting |
| | cell death ↑ x2 | binding protein ↓ x3 | protein 1, isoform |
| | Bax γ ↑ x1.5 | Apotosis-associated | BNIP1-a ↓ x1.5 |
| | BCL2/adenovirus E1B | tyrosine kinase ↑ x3.5 | Neuro-blastoma |
| | 19 kD-interacting protein | | apoptosis-related |
| | 3 ↑ x1.5 | | RNA binding |
| | Programmed cell | | protein ↓ x3 |
| | death 6 ↑ x1.5 | | |
| | Neuroblastoma- | | |
| | amplified protein | | |
| | ↑ x1.5 | | |

TABLE 3
Pasireotide Gene Expression Profiling (Continued)

| CLASS | KIDNEY | LIVER | SPLEEN | THYROID |
|---|---|---|---|---|
| SIGNAL | | | | |
| TRANSDUCTION | | | | |
| 1) Phophatidylinositol | PI-3-kinase, | PI transfer | PI-3-kinase, | IP3 |
| and related | catalytic, α | protein, β ↓ x1.5 | catalytic, α | receptor |
| pathways/PKC, | polypeptide ↓ x3 | 1-PI-4- | polypeptide ↓ x2.5 | type 3 |
| phospholipases | | phosphate 5- | PI-3-kinase, | ↓ x1.5 |
| | | kinase isoform C | class 3 ↑ x2 | PLC, γ 1 |
| | | ↓ x2 | PLA2 ↑ x2 | (formerly |
| | | Glycosylphosphatidylinositol | PKC, α binding | subtype |
| | | specific | protein ↑ x2 | 148) ↓ x2 |
| | | phospholipase D1 | IP-4- | PIP 5- |
| | | ↓ x1.5 | phosphatase, type | phosphatase |
| | | PKC, ι ↓ x1.5 | 1, isoform b | type IV ↓ |
| | | PLC, γ 1 | ↑ x2 | x3 |
| | | (formerly subtype | PI-3-kinase, | DAG 1 |
| | | 148) ↑ x2 | class 2, β | kinase, α |
| | | PKC substrate | polypeptide | (80 kD) |
| | | 80K-H ↑ x1.5 | ↑ x1.5 | ↓ x4 |
| | | PLA2, group IIA | Phosphatidylinositol | |
| | | (platelets, synovial | glycan, | |
| | | fluid) ↑ x5.5 | class B | |
| | | PI transfer | ↑ x1.5 | |
| | | protein ↑ x3.5 | | |
| | | Nck, Ash and | | |
| | | PLC γ binding | | |
| | | protein NAP4 | | |
| | | ↑ x3.5 | | |
| | | DAG kinase, α | | |
| | | (80 kD) ↑ x3 | | |
| | | DAG kinase, δ | | |
| | | (130 kD) | | |
| | | ↑ x1.5 | | |
| | | IP 5- | | |
| | | phosphatase ↑ x2 | | |
| 2) Other | PP 3 | Calcium/ | Nuclear factor of | FKBP- |
| calcium/ | (formerly | calmodulin- | activated T-cells, | associated |
| calcineurin/ | 2B), catalytic | dependent protein | cytoplasmic, | protein ↓ x2.5 |
| calmodulin | subunit, β | kinase kinase 2, β | calcineurin- | Calmodulin- |
| dependent | isoform | ↑ x3 | dependent 1 ↓ x5 | dependent PK |
| pathways and | (calcineurin | Calmodulin 2 | Calmodulin 1 | IV (CaM-kinase |
| associated | A β) ↓ x2 | (phosphorylase | (phosphorylase | IV) ↓ x7.5 |
| proteins | Calmodulin | kinase, δ) ↑ x2 | kinase, δ) ↑ x2 | Calcium/ |
| | 3 (phosphorrylase | Receptor | Calmodulin 2 | calmodulin- |
| | kinase, δ) | (calcitonin) activity | (phosphorylase | dependent PK |
| | ↓ x1.5 | modifying protein | kinase, δ) ↑ x1.5 | IV ↓ x7.5 |
| | Calcium/ | 1 precursor ↑ x1.5 | Calcium/ | c-AMP |
| | calmodulin- | | calmodulin- | responsive |
| | dependent | | dependent protein | element binding |
| | protein | | kinase (CaM kinase) | protein 1 ↓ x2 |
| | kinase | | II β ↑ x2 | Calcium/ |
| | kinase 2 β | | | calmodulin |
| | ↑ x1.5 | | | dependent |
| | | | | protein kinase 1 |
| | | | | ↓ x2 |
| 3) Ras/MAPK | Ras | Ras association | Rho/rac guanine | Ras homolog |
| kinase/ERK kinase | suppressor | (RalGDS/AF-6) | nucleotide exchange | gene family, |
| related pathways | protein 1 | domain family 1 | factor (GEF) 2 | member B |
| and adaptor | Rho | Rho GDP | Rab | Ras-GTPase |
| proteins | GTPase | dissociation | geranylgeranyl- | activating |
| | activating | inhibitor (GDI) γ | transferase | protein |
| | protein 4 | Rab | Human rho GDP- | SH3 domain- |
| | MAPKK 5 | geranylgeranyl- | dissociation inhibitor | binding protein 2 |
| | Rho | transferase, | 2 (IEF 8120) | Rho- |
| | GTPase | α subunit | SHP2 interacting | associated, |
| | activating | MAPK 10 | transmembrane | coiled-coil |
| | protein 5 | RAB13, member | adaptor | containing |
| | RAB4, | RAS oncogene | RAS p21 protein | protein kinase 1 |
| | member | family | activator (GTPase | RAD54 (S. cerevisiae)- |
| | RAS | Ras homolog | activating protein) 1 | like |
| | oncogene | gene family, | SH3 domain | RAB6, |
| | family | member G | binding glutamic | member RAS |
| | RAB | (rho G) | acid-rich protein like | oncogene family |
| | interacting | RAB2, member | Neuronal shc | RAB5A, |
| | factor | RAS oncogene | MAPKK 1 | member RAS |
| | Related | family | MAKKK 5 | oncogene family |
| | RAS viral | MAPK 1 | RAB11B, member | MAPKK 4 |
| | (r-ras) | C-src tyrosine | of RAS oncogene | SH3 domain |
| | oncogene | kinase | family | binding glutamic |
| | homolog | MAPK 14 | GTPase | acid-rich protein |
| | RAP2A, | Rho GTPase- | RAB5B, member | MAPK 8 |
| | member of | activating | RAS oncogene family | Adaptor |
| | RAS | protein 1 | MAPKAPK 2 | protein with |
| | oncogene | MAPKK 1 | MAPK 6 | pleckstrin |
| | family | ATP(GTP)- | Ras-related C3 | homology and |
| | Human | binding protein | botulinum toxin | src homology 2 |
| | rho GDP- | RAB interacting | substrate 1 isoform | domains |
| | dissociation | factor | Rac 1b | SHP2 |
| | inhibitor | MAPK 6 | RAB1, member ras | interacting |
| | MAPKK 1 | KAPKK 5 | oncogene family | transmembrane |
| | | RAB 30, | RAP1A, member of | adaptor |
| | | member RAS | ras oncogene family | Ras homolog |
| | | oncogene family | MAPKKKK | gene family, |
| | | RAB 4, member | RAS guanyl | member H |
| | | RAS oncogene | releasing protein 2 | RaP2 |
| | | family | (calcium and DAG- | interacting |
| | | MAPKK 13 | regulated) | protein 8 |
| | | MAP/ERK | Grb2-associated | RAB5C, |
| | | kinase kinase 4, | binder 2 | member RAS |
| | | isoform a | | oncogene family |
| | | | | Grb2- |
| | | | | associated |
| | | | | binder 2 |
| 4) JAK/STAT | STAT 2, 113 kD | STAT 1, 91kδ | JAK 3 | JAK 1 |
| pathway and | STAT 5B | | | STAT 6, |
| related kinases | | | | IL-4 |
| | | | | induced |
| | | | | Protein |
| | | | | inhibitor of |
| | | | | STATX |
| | | | | STAT 1, |
| | | | | 91kδ |
| | | | | STAT 3 |
| | | | | (acute- |
| | | | | phase |
| | | | | response |
| | | | | factor) |
| 5) Protein tyrosine | PP 1, regulatory | PTP | Dual specificity | PTP σ |
| phosphatases/other | subunit 7 | PP 2 (formerly | phosphatase 8 | Phosphatase |
| phosphatases | PP 1A (formerly | 2A), regulatory | Myosin | and |
| | 2C), Mg- | subunit A (PR 65), | phosphatase | tensin |
| | dependent, | α Isoform | target subunit 1 | homolog 2 |
| | α isoform | PP2A subunit-α | Dual specificity | PP 5, |
| | PTP | PTP, non | phosphatase 9 | catalytic |
| | PP 2A, | receptor type 1 | PTP, non- | subunit |
| | regulatory subunit | PTP, non | receptor type 1 | PTP, |
| | B′ (PR 53) | receptor type | PP 1, regulatory | non- |
| | PP 2A, | substrate 1 | (inhibitor) | receptor |
| | regulatory | PP 6, catalytic | subunit 8 | type 6 |
| | subunit-β | subunit | PTP, receptor | PP 1A |
| | PTP, non- | PTP, receptor | type, N | (formerly |
| | receptor type 1 | type, C | PTP type IVA, | 2C), Mg- |
| | PTP type IVA, | PP 1, regulatory | member 3 | dependent, |
| | member 3 | (inhibitor) | PP 5, catalytic | α isoform |
| | PP5, catalytic | subunit 5 | subunit | PTP, |
| | subunit | PTP, receptor | PTP σ | receptor |
| | | type, f polypeptide | | type, C |
| | | (PTPRF), | | PTP, |
| | | interacting protein | | receptor |
| | | (liprin), α 1 | | type, N |
| | | | | Phosphatidic |
| | | | | acid |
| | | | | phosphatase |
| | | | | type 2A |
| | | | | PTP, |
| | | | | receptor |
| | | | | type, A |
| 6) Other protein | Receptor | Protein kinase, | Serine/threonine | Serine |
| kinases and | tyrosine kinase | cAMP-dependent, | kinase 14 α | kinase |
| associated binding | Protein kinase | catalytic, | Serine/threonine | Serine/ |
| proteins | Serine/threonine | inhibitor α | kinase | threonine |
| | kinase 3 | Tyrosine | Protein kinase, | kinase 25 |
| | SNF1-like | kinase 2 | AMP-activated, γ 1 | Serine/ |
| | protein kinase | Protein kinase | non-catalytic | threonine |
| | Serine/threonine | PTK2 protein | subunit | protein kinase |
| | kinase 9 | tyrosine kinase 2 | Protein kinase, | Ste-20 |
| | Membrane- | Ribosomal | cAMP-dependent, | related kinase |
| | associated kinase | protein S6 kinase, | catalytic inhibitor α | Ribosomal |
| | Ser-Thr protein | 90kδ, | Dual-specificity | protein S6 |
| | kinase related to | polypeptide 3 | tyrosine-(Y)- | kinase, 90kδ, |
| | the myotonic | Protein kinase, | phosphorylation | polypeptide 3 |
| | dystrophy protein | cAMP-dependent, | regulated kinase | Serine/ |
| | kinase | catalytic, γ | 1A | threonine |
| | cAMP- | Serine threonine | Dual-specificity | kinase 13 |
| | dependent protein | protein kinase | tyrosine-(Y)- | (aurora/IPL1- |
| | kinase | | phosphorylation | like) |
| | RI-β regulatory | | regulated kinase 2 | Protein- |
| | subunit | | isofom 1 | tyrosine |
| | Ribosomal | | Serine/threonine | kinase |
| | protein S6 kinase, | | kinase 19 | Membrane- |
| | 90kδ, | | | associated |
| | polypeptide 4 | | | kinase |
| | Serine/threonine | | | Dual- |
| | kinase 25 | | | specificity |
| | Fms-related | | | tyrosine-(Y)- |
| | tyrosine kinase 3 | | | phosphorylation |
| | | | | regulated |
| | | | | kinase 2 |
| | | | | isoform 1 |
| 7) Adenylate/ | Natriuretic | Natriuretic | | Adenylate |
| guanylate | peptide receptor | peptide receptor A/ | | cyclase |
| cyclases and | A/guanylate | guanylate cyclase | | activating |
| related pathways | cyclase A | A (atrionatriuretic | | polypeptide |
| | (atrionatriuretic | peptide receptor | | precursor |
| | peptide receptor | A) ↑ x6 | | ↓ x1.5 |
| | A) ↑ x2 | Adenylyl cyclase- | | |
| | | associated protein | | |
| | | ↑ x1.5 | | |
| CELL SURFACE | | | | |
| RECEPTORS | | | | |
| 1) G-protein | Guanine | G protein- | Guanine | Guanine |
| coupled receptors | nucleotide binding | coupled | nucleotide | nucleotide binding |
| and related | protein (G protein), | receptor 12 | binding | protein (G protein), |
| binding proteins/G | β polypeptide 1 | G protein- | protein 11 | α 15 (Gq class) |
| proteins | G protein- | coupled | Guanine | G α inhibiting |
| | coupled | receptor kinase | nucleotide | activity polypeptide |
| | receptor 20 | G protein- | binding protein | 3 interacting |
| | G protein- | receptor | (G protein), | protein |
| | coupled receptor 9 | coupled 35 | β polypeptide 3 | Regulator of G |
| | G protein- | G protein- | G protein- | protein signalling |
| | coupled | coupled | coupled | Guanine |
| | receptor 39 | receptor 3 | receptor 56 | nucleotide binding |
| | G protein- | G protein- | Regulator of | protein 11 |
| | coupled receptor | receptor | G-protein | G protein- |
| | kinase | coupled 39 | signalling 9 | coupled receptor 3 |
| | G protein- | Regulator of | Guanine | G protein- |
| | coupled | G-protein | nucleotide | coupled receptor |
| | receptor 15 | signalling 6 | binding protein | kinase 1 |
| | Guanine | Coagulation | (G protein), α 11 | Ca++-sensing |
| | nucleotide binding | factor II | (Gq class) | receptor |
| | protein (G protein), | (thrombin) | G protein- | (hypocalcinuric |
| | β polypeptide 2 | receptor-like 1 | coupled | hypocalcaemia 1, |
| | G protein- | precursor | receptor 35 | severe neonatal |
| | coupled | | Angiotensin | hyperparathyroidism) |
| | receptor 35 | | receptor- | G protein- |
| | SSTR 3↑ x3 | | like 1 ↑ x1.5 | coupled receptor, |
| | SSTR 2↑ x2 | | SSTR2 ↑ x2 | family C, group 5, |
| | | | GDP | member B |
| | | | dissociation | Guanine |
| | | | inhibitor | nucleotide binding |
| | | | | protein 11 |
| | | | | 5-hydroxy- |
| | | | | tryptamine 7 |
| | | | | receptor isoform b |
| | | | | Developmentally |
| | | | | regulated |
| | | | | GTP-binding |
| | | | | protein 2 |
| | | | | Endothelial |
| | | | | differentiation- |
| | | | | related factor 1 ↑ |
| 2) Growth factors, | GFR-bound | Fms-related | EGF (β- | COL1A1 and |
| their receptors and | protein 7 ↑ x2 | tyrosine kinase | urogastrone) ↓ x2 | PDGFB fusion |
| related binding | GFR-bound | 1(VEGF/vascular | TGF induced | transcript ↓ x6 |
| proteins | protein 14 ↑ x2 | permeability factor | protein ↑ x1.5 | EGF-like |
| | CSF-1 receptor, | receptor) ↓ x1.5 | VEGF ↑ x1.5 | module |
| | formerly | GFR-bound | PDGF α | containing, |
| | McDonough feline | protein 2 ↓ x2.5 | polypeptide ↑ x2.5 | mucin-like, |
| | sarcoma viral (v- | Bone-derived GF | PDGFR, α | hormone |
| | fms) oncogene | ↑ x3 | polypeptide ↑ x1.5 | receptor-like |
| | homolog ↑ x2.5 | Growth | TGFβ receptor III | sequence 1 |
| | IL-7 R precursor | differentiation | (betaglycan, | ↓ x5 |
| | ↑ x2 | factor 1 ↑ x3 | 300 kD) ↓ x1.5 | PDGFR α |
| | PDGFR, α | TGF, β1 ↓ x1.5 | HGF activator | ↓ x3 |
| | polypeptide ↑ x1.5 | TGFβR III | inhibitor precursor | Fms-related |
| | TGF, β 1 ↑ x1.5 | (betaglycan, | ↑ x1.5 | tyrosine kinase |
| | | 300 kδ) ↓ x2 | | 1 (VEGF/ |
| | | EGF (β- | | vascular |
| | | urogastrone) | | permeability |
| | | ↓ x2.5 | | factor receptor) |
| | | EGFR (avian | | ↓ x2 |
| | | erythroblastic | | PDGF- |
| | | leukaemia viral (v- | | associated |
| | | erb-b) oncogene | | Protein ↑ x2 |
| | | homolog) ↑ x2 | | PDGFR β |
| | | Butyrate | | ↑ x2 |
| | | response factor 2 | | Cadherin 13, |
| | | (EGF-response | | H-cadherin |
| | | factor 2) ↑ x2 | | (heart) |
| | | FGFR2 | | ↑ x2 |
| | | (bacteria- | | |
| | | expressed kinase, | | |
| | | keratinocyte | | |
| | | growth factor | | |
| | | receptor, | | |
| | | craniofacial | | |
| | | dysplasia) ↑ x2 | | |
| | | TGFβ activated | | |
| | | kinase-binding | | |
| | | protein 1 ↑ x2.5 | | |
| | | GCSF ↑ x3.5 | | |
| | | EGF-like repeats | | |
| | | and discoidin I-like | | |
| | | domains 3 ↓ x1.5 | | |
| | | PDGFR, α | | |
| | | polypeptide ↑ x2 | | |
| | | PDGF, α | | |
| | | polypeptide ↑ x1.5 | | |
| 3) Glutamate | Glutamate | Glutamate | | Glutamate |
| receptor and | receptor | receptor, | | receptor, |
| related binding | metabotropic 2 | metabotropic 4 | | metabotropic 2 |
| proteins | precursor ↓ x3 | ↑ x1.5 | | precursor ↓ 3.5 |
| ATP- | Solute carrier | Ca++ channel, | K+ voltage-gated | K+ voltage- |
| DEPENDENT | family 6 | voltage- | channel, shaker- | gated channel, |
| TRANSPORT | (neurotransmitter | dependent, P/Q | related subfamily, | shaker-related |
| PROTEINS | transporter, | type, alpha 1A | member 3 ↓ x3 | subfamily, |
| Ion channels and | creatin), member 8 | subunit ↓ x2.5 | Solute carrier | member 3 |
| related pathways | ↓ x3 | ATPase, H+/K+ | family 9 (Na+/H+ | ↓ x4.5 |
| | Na+ channel, | exchanging, beta | exchanger) | ATPase, |
| | nonvoltage-gated | polypeptide ↓ x2 | isoform 3 | Ca++ |
| | 1, β (Liddle | Solute carrier | regulatory factor 1 | transporting, |
| | syndrome) ↓ x2 | family 9 (sodium/ | ↑ x10.5 | cardiac muscle, |
| | Ca++ channel, | hydrogen | ATPase, Na+/K+ | fast twitch 1 |
| | voltage- | exchanger), | transporting, β 1 | ↓ x4.5 |
| | dependent, α 1H | isoform 3 | polypeptide ↑ x2.5 | |
| | subunit ↑ x2 | regulatory factor 1 | K+ large | |
| | K+ voltage-gated | ↑ x11.5 | conductance | |
| | channel, Shaw- | Solute carrier | Ca++-activated | |
| | related subfamily, | family 11 | channel, subfamily | |
| | member 3 ↑ x2.5 | (Na+/phosphate | M, β member 1 | |
| | Solute carrier | symporters), | ↑ x2.5 | |
| | family 9 (Na+/H+ | member 1 ↑ x2.5 | Ca++ channel, | |
| | exchanger) | | voltage- | |
| | isoform 3 | | dependent, α 2/δ | |
| | regulatory factor 1 | | subunit 2 | |
| | ↑ x2 | | ↑ x1.5 | |
| | | | Ca++ channel, | |
| | | | voltage- | |
| | | | dependent, α 2/δ | |
| | | | subunit 1 ↑ x2 | |
| CELL BIOLOGY/ | | | | |
| SPECIALIZED | | | | |
| FUNCTIONS | | | | |
| 1) Neuromediators/ | δ sleep inducing | GABA (A) | Dopamine | GABA (A) |
| neuromodulators | peptide, | receptor, γ2 | receptor D4 ↓ x2 | receptor, |
| and related | immunoreactor↑ | precursor ↑ x4 | Adrenergic α-2C- | γ 2 precursor |
| pathways | x2.5 | Dopamine | receptor | ↓ x1.5 |
| | Opioid receptor, | receptor D2 ↑ x3.5 | ↓ x2 | Brain |
| | δ1 ↑ x2.5 | GABA(A) | β adrenergic | cannabinoid |
| | GABA (A) | receptor- | receptor | receptor 1 |
| | receptor, γ2 | associated protein | kinase 1 ↑ x3 | ↓ x2 |
| | precursor ↑ x2 | ↑ x1.5 | | GABA (B) |
| | Acetylserotonin | Dopamine | | receptor 1, |
| | O-methyl | receptor D3 ↑ x2 | | isoform a |
| | transferase-like | δ sleep inducing | | precursor |
| | ↓ x3 | peptide, | | ↑ x2.5 |
| | LIF (cholinergic | immunoreactor | | Cannabinoid |
| | differentiation | ↑ x2.5 | | receptor 2 |
| | factor) ↑ x2 | 5-hydroxytrypt | | (macrophage) |
| | Dopamine | amine (serotonin) | | ↑ x3 |
| | receptor D2 | receptor 6 ↑ x2.5 | | Phosphatidyl |
| | ↑ x4.5 | | | ethanolamine |
| | | | | N-methyl- |
| | | | | transferase |
| | | | | ↑ x2 |
| | | | | Adrenergic, α- |
| | | | | 2C-, receptor |
| | | | | ↓ x2.5 |
| 2) Pancreatic/ | | | Gastric inhibitory | Cholecystokinin |
| gastro-intestinal | | | polypeptide | B receptor ↓ x3 |
| secretions and | | | receptor ↑ x2 | Gastric |
| related pathways | | | | inhibitory |
| | | | | polypeptide 1 |
| | | | | receptor ↓ x2 |
| 3) Hormones and | Angiotensin | Insulin promoter | PTHR 1 ↓ x2.5 | TSHR ↓ x2 |
| related pathways | receptor | factor 1, | Arginine | IGF-1 ↓ x1.5 |
| | 1B ↓ x1.5 | homeodomain | vasopressin | Solute carrier |
| | Glucocorticoid | transcription factor | receptor 1B ↑ x2 | family 21 (PG |
| | receptor DNA | ↓ x1.5 | IGFBP6 ↑ x2.5 | transporter), |
| | binding factor 1 | IGF-2 ↓ x2.5 | IGF-1 ↑ x1.5 | member 2 ↑ x2 |
| | ↓ x4.5 | Corticosteroid | | |
| | Insulin receptor | binding globulin | | |
| | ↓ x3 | precursor ↑ x2 | | |
| | THR, α (avian | THR interacting | | |
| | erythroblastic | protein 15 ↑ x1.5 | | |
| | leukaemia viral (v- | IGFBP2 ↑ x2 | | |
| | erb-a) oncogene | Arginine | | |
| | homolog) ↑x1.5 | vasopressin | | |
| | Arginine | receptor 2 ↑ x2.5 | | |
| | vasopressin | THR sulfo | | |
| | (neurophysin II, | transferase ↑ x2.5 | | |
| | antidiuretic | Glucacon | | |
| | hormone, diabetes | receptor ↑ x5 | | |
| | insipidus, | | | |
| | neurohypophyseal) | | | |
| | ↑ x2 | | | |
| | Vasopressin- | | | |
| | activated calcium- | | | |
| | mobilizing | | | |
| | receptor-1 ↓ x2 | | | |
| | Corticotropin | | | |
| | releasing hormone | | | |
| | receptor type 2 | | | |
| | beta isoform | | | |
| | ↑ x1.5 | | | |
| | IGF-2 ↓ x2 | | | |
| | IGF-1 ↓ x2.5 | | | |
| | IGFBP2 ↑ x1.5 | | | |
| | THR-associated | | | |
| | protein, 240kδ | | | |
| | subunit ↓ x1.5 | | | |
| | THR binding | | | |
| | protein ↑x1.5 | | | |
| | PG-endo- | | | |
| | peroxide synthase | | | |
| | 1 (prostaglandin | | | |
| | G/H synthase and | | | |
| | cyclooxygenase) | | | |
| | ↓ x3.5 | | | |
| | Adrenomedullin | | | |
| | ↑ x1.5 | | | |
| | SSTR 3↑ x3 | | | |
| | SSTR 2↑ x2 | | | |
| 4) Cytoskeleton | VWF | | Vasodilatator- | VWF |
| and associated | precursor ↑ x2 | | stimulated | precursor ↑ x2 |
| proteins | | | phosphoprotein ↑ x2 | Thrombo- |
| 5) Enzymes | | | | spondin 2 ↑ x2 |
| | | | | Pro-platelet |
| | | | | basic protein |
| | | | | (includes |
| | | | | platelet basic |
| | | | | protein, β- |
| | | | | thrombo- |
| | | | | globulin, |
| | | | | connective |
| | | | | tissue- |
| | | | | activating |
| | | | | peptide III, neu) |
| | | | | 2 ↑ x3.5 |
| IMMUNITY | TNF, α- | TNF-α converting | IFN-inducible RNA- | LTB4 receptor |
| | induced | enzyme ↓ x2 | dependent protein | (chemokine |
| | protein 3 | IFN-stimulated | kinase | receptor-like 1) |
| | ↓ x1.5 | protein, 15 kDa ↓ x2 | ↑ x2.5 | ↓ x1.5 |
| | IRF5 ↑ x2 | IFN-related | TNF (cachectin) | IL2-inducible T- |
| | Putative | developmental | ↑ x2 | cell kinase ↓ x12 |
| | chemokine | regulator 2 ↓ x1.5 | TNF (ligand) | P56lck ↓ x18 |
| | receptor; GTP- | IFN-inducible RNA- | superfamily, member | RAG1 ↓ x18 |
| | binding protein | dependent protein | 13 ↑ x2.5 | IFNγ responsive |
| | ↑ x2 | kinase ↑ x4 | IL 1 receptor-like 1 | transcript ↓ x1.5 |
| | TNFR | IL2 R, γ chain, | ↑ x2 | SH2 domain |
| | superfamily, | precursor ↑ x2.5 | IFNγ responsive | protein 1A, |
| | member 12 | IFN regulatory | transcript | Duncan's disease |
| | ↑ x2 | factor 5 ↑ x2.5 | ↑ x2 | (lymphoproliferative |
| | | Bruton agamma | LTb4 (chemokine | syndrome) |
| | | globulinaemia | receptor-like 1) ↑ x3 | ↓ x7.5 |
| | | tyrosine kinase | Putative chemokine | CD2 antigen |
| | | ↑ x1.5 | receptor; GTP- | (p50), sheep red |
| | | B lymphoid tyrosine | binding protein ↑ x2.5 | blood cell receptor |
| | | kinase | IFNγ receptor 2 | ↓ x7.5 |
| | | ↑ x3.5 | (IFNγ transducer 1) | TCR ζ chain |
| | | IFN γ responsive | ↑ x1.5 | precursor ↓ x5.5 |
| | | transcript ↑ x1.5 | TNFR superfamily, | RAG2 ↓ x5 |
| | | TNF (ligand) | member 12 ↑ x1.5 | Signalling |
| | | superfamily, member | IL-8 receptor type B | lymphocytic |
| | | 10 ↑ x1.5 | ↑ x1.5 | activation |
| | | IFN-induced | IFN regulatory | molecule ↓ x4.5 |
| | | leucine zipper protein | factor 2 ↑ x3.5 | Flt3 ligand ↓ x4.5 |
| | | ↑ x1.5 | | Lymphocyte |
| | | | | specific protein |
| | | | | tyrosine kinase |
| | | | | ↓ x4 |
| | | | | Chemokine |
| | | | | (C-X-C motif), |
| | | | | receptor 4 |
| | | | | (fusin) ↓ x3 |
| | | | | Transcription |
| | | | | factor 7 (T-cell |
| | | | | specific, HMG- |
| | | | | box) ↓ x16.5 |
| | | | | IL9 receptor |
| | | | | ↓ x2 |
| | | | | RANTES |
| | | | | ↑ x10.5 |
| | | | | CD2 antigen |
| | | | | (cytoplasmic |
| | | | | tail)-binding |
| | | | | protein 2 |
| | | | | ↑ x3.5 |
| | | | | IFNγ- |
| | | | | inducible |
| | | | | protein 30 |
| | | | | ↑ x3.5 |
| | | | | IFNα- |
| | | | | inducible |
| | | | | protein 27 ↑ x3 |
| | | | | TNF (ligand) |
| | | | | superfamily |
| | | | | member 10 |
| | | | | ↑ x2 |
| CELL CYCLE | Cdk (CDC2-like) | Cdki 2D (p19, | Cyclin I ↓ x1.5 | Cdc-like 5 |
| | ↓ x2 | inhibits CDK4) | S-phase kinase- | (cholinesterase- |
| | Growth arrest- | ↓ x2.5 | associated protein | related cell |
| | specific 6 ↑ x1.5 | Cdk like-2 ↓ x2 | 1A (p19A) ↑ x2 | division |
| | Cdk 5, regulatory | Cdk 5, regulatory | Cdk 6 ↑ x3 | controller) |
| | subunit 2 (p39) | subunit 1 ↑ x2.5 | Cdk 5, regulatory | ↓ x2 |
| | ↑ x1.5 | Cdki1A | subunit 1 (p35) | Cdk (CDC2- |
| | CDC37 (cell | (p21/Cip1) ↑ x6 | ↑ x2 | like) ↓ x9 |
| | division cycle 37, | Cdk like-2 ↓ x2 | Cyclin D2 ↑ x1.5 | Cdk 5, |
| | S. cerevisiae, | | S-phase kinase- | regulatory |
| | homolog) ↑ x1.5 | | associated protein | subunit 1 (p35) |
| | Cdki 1A (p21, | | 1A ↑ x2 | ↓ x5 |
| | Cip 1) ↑ x5 | | Cdk 2 ↑ x1.5 | Growth |
| | | | Follistatin-like 1 | arrest-specific 1 |
| | | | ↑ x1.5 | ↓ x2.5 |
| | | | | Cyclin B2 |
| | | | | ↓ x2.5 |
| APOPTOSIS | Death effecter | BCL2-like 1 | Rb binding | Bcl-2 binding |
| | domain-containing | ↓ x3.5 | protein ↑ x2 | component 3 |
| | ↓ x3.5 | Rb 1 (including | Caspase 8, | ↓ x2 |
| | Fas/Apo-1/CD95 | osteosarcoma) | apoptosis-related | |
| | ↑ x1.5 | ↓ x2 | cysteine protease | |
| | | Death- | ↑ x2.5 | |
| | | associated protein 6 | TNF (cachectin) | |
| | | ↓ x2.5 | ↑ x2 | |
| | | Rb binding | TNF (ligand) | |
| | | protein ↑ x2.5 | superfamily, | |
| | | Rb-like 2 | member 13 ↑ x2.5 | |
| | | (p130)↑ x3 | Bcl-2 binding | |
| | | Fas-activated | component 3 | |
| | | serine/threonine | ↑ x2.5 | |
| | | kinase ↑ x1.5 | Death- | |
| | | | associated protein | |
| | | | ↑ x1.5 | |
| | | | Tumour protein | |
| | | | p53-binding | |
| | | | protein ↑ x1.5 | |
| | | | Rb-binding | |
| | | | protein 8 ↑ x1.5 | |
| | | | Programmed cell | |
| | | | death 10 ↑ x1.5 | |

These results show that several signal transduction pathways were affected. They included the phosphatidylinositol/PKC/phospholipases/calcium-calcineurin-calmodulin pathway, the Ras/MAPK kinase/ERK kinase dependent pathway, the JAK/STAT pathway, and adenylate/guanylate cyclases with their dependent pathways. The changes for the cell surfaces receptors included numerous G-protein coupled receptors, receptors for growth factors and glutamate receptors. The changes in ATP-dependent transport proteins involved ion channels and associated proteins. The compound also affected neuromediators/neuromodulators, pancreatic and gastrointestinal secretions, hormones, cytoskeletal proteins and enzymes/catalysts.

Examples of genes reflecting several SSTR signalling pathways in the pituitary are shown in TABLE 4. Selected genes from the primary gene lists were produced by a succession of filtering and statistical algorithms (t-test: p value: 0.05). The numerical values correspond to the AvgDiff (see above) of the relevant probe set for each experiment with the range of observed values between brackets. Of particular interest in this analysis were the transcript level changes for molecules known to be closely associated with the binding of the natural peptides, SST-14 and SST-28, to the SSTRs.

TABLE 4
Examples of Genes Reflecting Several SSTR Signalling Pathways in the Pituitary

| | | Pasireotide |
|---|---|---|
| GENES | CONTROL | (0.1 mg/animal/14 day) |
| SIGNAL TRANSDUCTION | | |
| 1) Phophatidyl inositol and related | | |
| pathways/PKC, phospholipases | | |
| IP-4-phosphatase, type 1, isoform b | 296 (241 to 342) | 177 (107 to 232) |
| PI-3-kinase, catalytic, δ polypeptide | 91 (45 to 146) | 34 (20 to 67) |
| PI-3-kinase, catalytic, α polypeptide | 72 (26 to 135) | 21 (20 to 24) |
| PI transfer protein, β | 125 (93 to 187) | 42 (34 to 50) |
| PKC inhibitor | 2,351 (2,135 to 2,755) | 3,333 (2,339 to 3,878) |
| PLC, γ 1 (formerly subtype 148) | 111 (100 to 131) | 40 (20 to 63) |
| PKC inhibitor | 2,351 (2,1345 to 2,755) | 3,332 (2,339 to 3,878) |
| 2) Ras/MAPK kinase/ERK kinase related | | |
| pathways and adaptor proteins | | |
| MAPKKK5 | 171 (148 to 207) | 278 (221 to 351) |
| Rab geranylgeranyltransferase, α subunit | 164 (152 to 173) | 104 (70 to 172) |
| Rab geranylgeranyltransferase, βsubunit | 230 (187 to 250) | 284 (246 to 374) |
| SHB adaptor protein (a Src homology 2 | 112 (43 to 190) | 38 (20 to 55) |
| protein) | | |
| RAB 5C, member RAS oncogene family | 72 (20 to 138) | 162 (109 to 212) |
| 3) Protein tyrosine phosphatases/other | | |
| phosphatases | | |
| Dual specificity phosphatase 8 | 493 (344 to 625) | 170 (67 to 238) |
| Phosphatase and tensin homolog (mutated in | 129 (58 to 228) | 36 (20 to 63) |
| multiple advanced cancers 1) | | |
| PTP, receptor type, T | 58 (41 to 78) | 101 (48 to 129) |
| PP 1, regulatory (inhibitor) subunit 5 | 20 | 75 (60 to 90) |
| 4) Adenylate/guanylate cyclases and related | | |
| pathways | | |
| Soluble adenylyl cyclase | 54 (51 to 57) | 22 (20 to 27) |
| CELL SURFACE RECEPTORS | | |
| 1) G-protein coupled receptors | | |
| SSTR3 | 22 (20 to 24) | 57 (20 to 90) |
| 2) Glutamate receptor and related binding | | |
| proteins | | |
| GLUR 2, precursor | 42 (20 to 86) | 59 (20 to 177) |
| ATP-DEPENDENT TRANSPORT PROTEINS | | |
| Ion channels and related pathways | | |
| ATPase, Na+/K+ transporting, β 3 polypeptide | 292 (246 to 353) | 610 (335 to 949) |
| ATPase, Na+/K+ transporting, α 2 (+) | 86 (52 to 130) | 184 (69 to 325) |
| polypeptide | | |
| ATPase, H+/K+ exchanging, α polypeptide | 128 (50 to 245) | 20 |
| K+ channel, subfamily K, member 3 (TASK) | 132 (69 to 188) | 26 (20 to 43) |
| K+ voltage-gated channel, Shab-related | 66 (20 to 112) | 22 (20 to 31) |
| subfamily, member 1 | | |
| Putative Ca++ transporting ATPase | 61 (38 to 98) | 101 (84 to 112) |
| CELL CYCLE | | |
| Core-binding factor, runt domain, α subunit 2; | 225 (113 to 343) | 491 (251 to 677) |
| translocated to, 1; cyclin D-related | | |
| Forkhead box O3A | 497 (447 to 553) | 257 (186 to 324) |
| Forkhead box H1 | 225 (113 to 343) | 117 (251 to 677) |
| Cyclin F | 198 (171 to 229) | 74 (48 to 132) |
| Cyclin D3 | 187 (173 to 201) | 338 (202 to 446) |
| S-phase response (cyclin-related) | 91 (88 to 97) | 129 (111 to 148) |
| Cell division cycle 25B | 40 (20 to 67) | 162 (134 to 187) |
| Cdk inhibitor 2C (p18, inhibits CDK4) | 81 (58 to 99) | 184 (140 to 229) |
| Cdk inhibitor 2D (p19, inhibits CDK4) | 198 (171 to 229) | 99 (83 to 118) |
| APOPTOSIS | | |
| BCL2-associated athanogene | 216 (207 to 231) | 318 (235 to 409) |
| BCL2-antagonist of cell death | 44 (33 to 47) | 69 (42 to 89) |
| Bax gamma | 258 (207 to 297) | 326 (221 to 448) |
| BCL2/adenovirus E1B 19 kD-interacting protein 3 | 342 (288 to 401) | 458 (388 to 526) |
| Programmed cell death 6 | 504 (443 to 547) | 635 (513 to 747) |
| Neuroblastoma-amplified protein | 178 (149 to 210) | 237 (201 to 258) |

The effects on the GH/IGF-1 and glucagon/insulin axes (Macaulay V M, Br. J. Cancer 65: 311-20 (1992); Pollak M N & Schally A V, Proc. Soc. Exp. Biol. Med. 217: 143-52 (1998)) were reflected in transcript level changes in several organs. The results are shown in TABLE 5. Beside the expected change in IGF-1 transcript level, there was an effect on IGF-2 as well (in the pituitary and kidneys) that might be useful as a biological marker of pasireotide activity if reflected in the blood. The genes were selected as above in TABLE 4.

TABLE 5
Example of genes reflecting the effects of pasireotide
on the GH/IGF and glucagon/insulin axes in different tissues

| | | PASIREOTIDE |
|---|---|---|
| | | (0.1 mg/ |
| ORGANS/GENES | CONTROL | animal/14day) |
| PITUITARY | | |
| IGF-2 | 126 (40 to 179) | 70 (20 to 150) |
| GR | 20 | 109 (51 to 215) |
| IGFBP, acid labile | 30 (20 to 49) | 83 (20 to 110) |
| subunit | | |
| SSTR3 | 22 (20 to 24) | 57 (20 to 90) |
| BROWN FAT | | |
| IGF-1 | 548 (279 to 810) | 389 (315 to 449) |
| IGFBP 4 | 1410 (916 to 2173) | 763 (429 to 1058) |
| IRS 2 | 48 (20 to 84) | 146 (80 to 222) |
| SSTR3 | 25 (20 to 52) | 194 (87 to 248) |
| PANCREAS | | |
| IGF-1 | 20 | 89 (20 to 298) |
| SSTR2 | 258 (205 to 366) | 156 (120 to 210) |
| KIDNEY | | |
| IR | 654 (187 to 1,187) | 196 (163 to 265) |
| IGF-2 | 117 (47 to 176) | 49 (20 to 39) |
| IGF-1 | 65 (24 to 103) | 25 (20 to 39) |
| IGFBP2 | 375 (211 to 625) | 563 (457 to 655) |
| SSTR 3 | 31 (20 to 69) | 82 (33 to 120) |
| SSTR 2 | 74 (20 to 153) | 126 (93 to 158) |
| LIVER | | |
| Insulin promoter | 89 (58 to 160) | 42 (23 to 52) |
| factor 1, | | |
| homeodomain | | |
| transcription factor | | |
| IGF-2 | 701 (403 to 961) | 269 (224 to 291) |
| IGFBP2 | 2,722 (1,321 to 3,363) | 4,476 (3,191 to 5,422) |
| GR | 44 (20 to 82) | 80 (70 to 360) |
| SPLEEN | | |
| IGFBP6 | 495 (130 to 982) | 1,043 (853 to 1,155) |
| IGF-1 | 72 (42 to 103) | 85 (52 to 125) |
| SSTR2 | 56 (20 to 83) | 93 (87 to 95) |
| THYROID | | |
| IGF-1 | 91 (20 to 179) | 58 (20 to 114) |

Other genes of interest affected by pasireotide were the transcript levels of growth factors (PDGF, FGF, EGF, TGFβ), their receptors and factors of angiogenesis (PDGF, VEGF, thrombospondin) involved in tumour growth and spreading (Woltering E A et al., New Drugs 15: 77-86 (1997)). Also reported for somatostatin and analogues, genes involved in immunity were changed, i.e. cytokines (IL-1, TNF, IFN), regulators of T and B cell genesis and function (CD2 antigen, IL-2 receptor, B-lymphoid tyrosine kinase, IL-2 inducible T cell kinase, p561ck, RAG1, TCRζ chain precursor, RAG2, FLT 3 ligand) (van Hagen P M et al. Eur. J. Clin. Invest. 24: 91-9 (1994)), as well as genes involved in blood pressure control and diuresis, i.e. atrial natriuretic peptide and its receptor guanylyl cyclase A, arginine vasopressin and its receptor (Aguilera G et al., Nature 292: 262-3 (1981); Aguilera G et al., Endocrinology 111: 1376-84 (1982); Ray C et al., Clin. Sci. (Lond) 84: 455-60 (1993); Cheng H et al., Biochem. J. 364: 33-9 (2002)). A specific gene involved in the control of fat storage is the adrenergic β3 receptor in brown fat (Bachman E et al., Science 297: 843-45 (2002)).

Protein products of the above genes are useful as surrogate markers of the biological activity of pasireotide, especially the findings for IGF-2 in the pituitary and kidneys.

To conclude, the gene profiling of monkey tissues treated with pasireotide at sub-therapeutic is a sensitive approach to identify signalling and effecter pathways known for somatostatin.

All references cited herein are incorporated herein by reference in their entirety and for all purposes to the same extent as if each individual publication or patent or patent application was specifically and individually indicated to be incorporated by reference in its entirety for all purposes. In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1-4. (canceled)

5. A method for monitoring the treatment of disorder of growth regulation, comprising the steps of:
(a) administering a somatostatin or a somatostatin analogue to the subject;
(b) obtaining the gene expression profile of the subject, wherein the gene expression profile comprises the gene expression pattern of one or more genes selected from the groups consisting of:
(i) a decrease in the gene expression in the pituitary of a gene selected from the group consisting of PKC inhibitor; MAPKKK5; rate geranylgeranyltransferase, o, subunit; SHE adaptor protein (a Src homology 2 protein); dual specificity phosphatase 8; phosphatase and tensin homolog; soluble adenylyl cyclase; ATPase. H+/K+ exchanging, or polypeptide; k+ channel, subfamily k, member 3 (TASK); K+ voltage gated channel, Shab-related subfamily, member 1; forkhead box 03A; forkhead box H1; cyclin F; cdk inhibitor 2D (pI9, inhibits CDK4) and combinations thereof;
(ii) an increase in the gene expression in the pituitary of a gene selected from the group consisting of IP-4-phosphatase, type 1, isoform b; PI-3-kinase, catalytic, polypeptide; PI-3-kinase, catalytic, a polypeptide; PI transfer protein, p; PLC, 1 (formerly subtype 148): Rab geranylgeranyltransferase it, subunit; RAB 5C, member RAS oncogene family; PTP, receptor type, T; PP 1, regulatory (inhibitor) subunit 5; SSTR3; GLUR 2, precursor; ATPase, Na+/K+ transporting, p3 polypeptide; ATPase, Na+/K+ transporting, a2 (+) polypeptide; putative Ca+ transporting ATPase; core binding factor, runt domain, a subunit 2; translocated to, 1: cyclin D-related; cyclin D3; S-phase response (cyclin-related); cell division cycle 25B; cdk inhibitor 2C (pI8, inhibits CDK4); BCL2-associated athanogene; BCL2-antagonist of cell death; Bax gamma: BCL2/adenovirus E1B I9 kD-interacting protein 3; programmed cell death 6; neuroblastoma-amplified protein and combinations thereof;
(iii) a decrease in the gene expression in the pituitary of the IGF-2 gene;
(iv) an increase in the gene expression in the pituitary of a gene selected from the group consisting of glucagon receptor (GR); IGFBP (acid labile subunit); SSTR3 and combinations thereof;
(v) a decrease in the gene expression in brown fat of a gene selected from the group consisting of IGF-1, IGFBP 4 and combinations thereof;
(vi) an increase in the gene expression in brown fat of a gene selected from the group consisting of IRS 2, SSTR 3 and combinations thereof;
(vii) a decrease in the gene expression in the pancreas of the IGF-1 gene;
(vii) an increase in the gene expression in the pancreas of the SSTR 2 gene;
(ix) a decrease in the gene expression in the kidney of a gene selected from the group consisting of IGF-1, IGF-2 and combinations thereof;
(x) an increase in the gene expression in the pancreas of a gene selected from the group consisting of IGFBP2, SSTR 3, SSTR 2 and combinations thereof;
(xi) a decrease in the gene expression in the liver of a gene selected from the group consisting of insulin promoter factor 1, homeodomain transcription factor, IGF-2 and combinations thereof;
(xii) an increase in the gene expression in the liver of a gene selected from the group consisting of IGFBP2, glucagon receptor (GR) and combinations thereof;
(xiii) a decrease in the gene expression in the spleen of a gene selected from the group consisting of IGFBP6, IGF-1, SSTR 2 and combinations thereof;
(xiv) an increase in the gene expression in the spleen of a gene selected from the group consisting of IGFBP2, glucagon receptor (GR) and combinations thereof:
(xv) an increase in the gene expression in the spleen of the IGF-1 gene; and
(xvi) a decrease in the gene expression of the IGF-2 gene as measured in a sample taken from the subject; and
(c) comparing the gene expression profile of the subject to whom the somatostatin or somatostatin analogue was administered to a biomarker gene expression profile indicative of efficacy of treatment by somatostatin or a somatostatin analogue, wherein a similarity in the gene expression profile of the subject to whom the somatostatin or somatostatin analogue was administered to the biomarker gene expression profile is indicative of efficacy of treatment with the somatostatin or somatostatin analogue.

6. (canceled)

7. The method of claim 5, wherein the somatostatin or somatostatin analogue is pasireotide.

8. The method of claim 5, wherein the subject is a mammal.

9. The method of claim 8, wherein the mammal is a primate.

10. The method of claim 9, wherein the primate is a cynomolgus monkey or a human.

11. The method of claim 5; wherein the biomarker gene expression profile is the baseline gene expression profile of the subject before administration of the somatostatin or somatostatin analogue.

12. The method of claim 5, wherein the biomarker gene expression profile is the gene expression profile or average of gene expression profiles of a vertebrate to whom somatostatin or a somatostatin analogue has been administered.

13-30. (canceled)

31. A method for determining whether a compound has a therapeutic efficacy similar to that of somatostatin or a somatostatin analogue, comprising the steps of:
(a) administering the compound to the subject;
(b) obtaining the gene expression profile of the subject, wherein the gene expression profile comprises the gene expression pattern of one or more genes, where the expression patterns of the one or more genes are a consequence of administration of the compound,
(c) comparing the gene expression profile of the subject to whom the compound, was administered to a biomarker gene expression profile indicative of efficacy of treatment by somatostatin or a somatostatin analogue; and
(d) then:
(i) determining that the compound has a therapeutic efficacy similar to that of somatostatin or a somatostatin analogue when the gene expression profile of the subject to whom the compound was administered is similar to the biomarker gene expression profile of a subject to whom somatostatin or a somatostatin analogue is administered; or
(ii) determining that the compound has a therapeutic efficacy different from that of somatostatin or a somatostatin analogue when the gene; expression profile of the subject to whom the compound was administered is different from the biomarker gene expression profile of a subject to whom somatostatin or a somatostatin analogue is administered.

32. The method of claim 31, wherein the somatostatin analogue is pasireotide.

33. The method of claim 31, wherein the subject is a mammal.

34. The method of claim 33, wherein the mammal is a primate.

35. The method of claim 34, wherein the primate is a cynomolgus monkey or a human.

36. The method of any one of claim 31, wherein the compound is administered to the subject at a sub-therapeutic dose.

37-41. (canceled)
